(19) United States
(12) Patent Application Publication
(10) Pub. No.: US 2026/0363919 A1
(43) Pub. Date: Nov. 19, 2026

(54) SYSTEM AND A DEVICE FOR COLLECTING MENSTRUAL BLOOD

(71) Applicant: LaTeria McGriff, Hampton, GA (US)

(72) Inventor: LaTeria McGriff, Hampton, GA (US)

(21) Appl. No.: 19/207,240

(22) Filed: May 13, 2025

(51) Int. Cl. A61F 13/20

(52) U.S. Cl. CPC A61F 13/2051

(57) ABSTRACT

A menstrual blood collection device is provided. The menstrual blood collecting device includes a compressed menstrual tampon. The compressed menstrual tampon includes an elongated generally rod-shaped absorption body having an insertion end and a withdrawal end and having a withdrawal string extending from the withdrawal end. The absorption body has at least one wave-shaped compression line extending continuously from the insertion end to the withdrawal end. The compressed menstrual tampon includes a rope that extends from the cotton insert then out the applicator is connected to a pad.

### CROSS-REFERENCES TO RELATED APPLICATIONS

N/A.

### FIELD OF THE TECHNOLOGY

The present disclosure generally relates to tampon designs for extra leakage protection and, more specifically, to menstrual “catching” along with extended strings into the cotton insert.

### DESCRIPTION OF THE RELATED ART

Traditionally women used other menstrual devices such as a pad, a tampon, and/or a menstrual cup. However, these devices failed to provide adequate leakage protection or comfort for its users.

Further, prior menstrual devices and/or tampon designs failed to apply extra leakage protection using pads for blood and other fluid absorption materials that allow for extended strings with a cotton insert.

What is needed is an is tampon designs for extra leakage protection configured to all for menstrual “catching” along with extended strings into the cotton insert.

### SUMMARY

In an embodiment, a system includes a menstrual blood collection device. The menstrual blood collecting device includes a compressed menstrual tampon. The compressed menstrual tampon includes an elongated generally rod-shaped absorption body having an insertion end and a withdrawal end and having a withdrawal string extending from the withdrawal end. The absorption body has at least one wave-shaped compression line extending continuously from the insertion end to the withdrawal end. The compressed menstrual tampon includes a rope that extends from the cotton insert then out the applicator is connected to a pad.

In another embodiment, a menstrual blood collection device is provided. The menstrual blood collecting device includes a compressed menstrual tampon. The compressed menstrual tampon includes an elongated generally rod-shaped absorption body having an insertion end and a withdrawal end and having a withdrawal string extending from the withdrawal end. The absorption body has at least one wave-shaped compression line extending continuously from the insertion end to the withdrawal end. The compressed menstrual tampon includes a rope that extends from the cotton insert then out the applicator is connected to a pad.

In still another embodiment, compressed menstrual tampon is provided. The compressed menstrual tampon includes an elongated generally rod-shaped absorption body having an insertion end and a withdrawal end and having a withdrawal string extending from the withdrawal end. The absorption body has at least one wave-shaped compression line extending continuously from the insertion end to the withdrawal end. The compressed menstrual tampon includes a rope that extends from the cotton insert then out the applicator is connected to a pad.

### BRIEF DESCRIPTION OF THE DRAWINGS

The present invention is described in detail below with reference to the attached drawing figures, which are incorporated by reference herein and wherein:

FIG. 1 is a block diagram illustrating an exemplary embodiment of the invention;

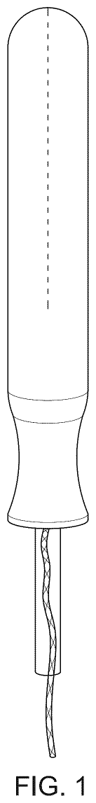

FIG. 2 is a block diagram in accordance with another exemplary embodiment of the invention;

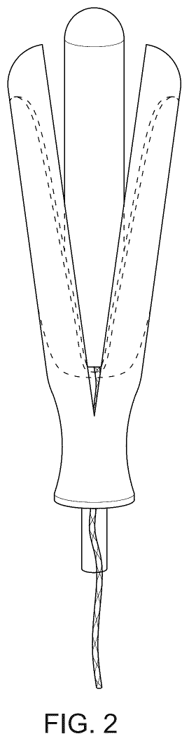

FIG. 3 is a system block diagram with a vehicle in accordance with another exemplary embodiment of the invention; and

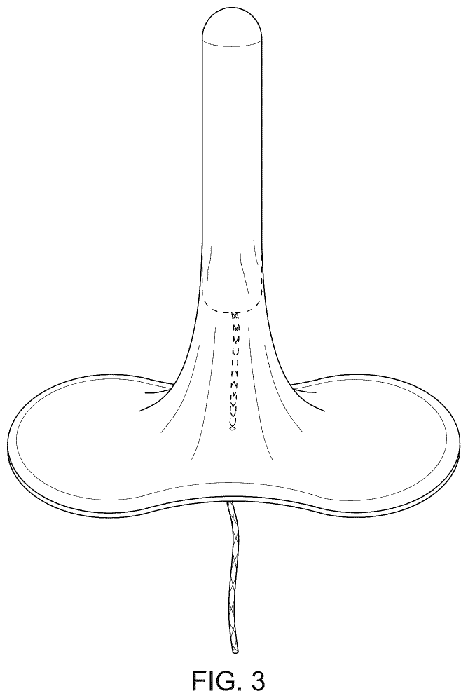

### DETAILED DESCRIPTIONS

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent application, which would still fall within the scope of the claims.

In an embodiment, the present invention includes a menstrual blood catching device designed to prevent leakage to one's undergarments. In some embodiments, the present invention may include a combination of a tampon and a pad. The present invention may be commercialized under the brand of Ella™. In other embodiments, the rope that extends from the cotton insert then out the applicator is connected to a pad. The applicator splits into two pieces for ease of removal. The present invention gives extra leakage security to its users.

In one embodiment, the present invention provides a funnel-shaped tampon, comprising an absorbent body configured with a narrow insertion end and a widened upper collection portion that collectively define a tapered or conical geometry. This shape facilitates easier insertion, improved anatomical conformity, and enhanced fluid channeling toward the absorbent core. The funnel configuration may include an optional internal cavity or layered structure to support directional absorption and expandability during use.

In another embodiment, the present invention includes a compressed menstrual tampon including an elongated generally rod-shaped absorption body having an insertion end and a withdrawal end and having a withdrawal string extending from the withdrawal end. The absorption body has at least one wave-shaped compression line extending continuously from the insertion end to the withdrawal end.

In yet another embodiment, the present invention includes an expandable bag catamenial tampon and spreading tampon applicators for such a tampon are disclosed. The catamenial tampon is in the form of a soft, conformable, hollow bag. The applicator can insert the tampon into the vaginal tract and spreading the tampon open within the vaginal cavity. The applicator has an applicator element with a leading end and a trailing end. The bag-like tampon is placed over the leading end of the applicator when the tampon is a flaccid initial configuration which defines an initial width, and the applicator is used to spread the tampon to a greater deployed width. In one embodiment, the applicator comprises tube having an interior and a flexible material which has one end that is formed into a flexible and expandable loop. The flexible loop is slidably mounted within the tube. The flexible loop is contained inside the tube until it is desired to deploy the tampon. When it is desired to deploy the tampon, the flexible loop with the tampon thereon is pushed out the expulsion end of the tube. The flexible loop expands laterally and spreads the tampon open within the vaginal cavity.

In still another embodiment, the present invention includes tampon for feminine hygiene that has an insertion end, a withdrawal end, a longitudinal axis, and an outer surface. The tampon is made of compressed fibrous material. The outer surface of the tampon includes a plurality of raised portions. Each of the raised portions includes a first width dimension x and a second width dimension y. The first width dimension x is greater than the second width dimension y.

In yet another embodiment, the present invention includes tampons including an elongate body and a removal string extending from a removal end of the elongate body. The tampons may include an absorbent material and a spiral flow path that directs menses across the elongate body. The spiral flow path includes at least one complete turnaround the longitudinal axis of the elongate body.

It should also be understood that, unless a term is expressly defined in this patent using the sentence, there is no intent to limit the meaning of that term, either expressly or by implication, beyond its plain or ordinary meaning, and such term should not be interpreted to be limited in scope based on any statement made in any section of this patent application (other than the language of the claims). To the extent that any term recited in the claims at the end of this patent application is referred to in this patent application in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

Many different arrangements of the various components depicted, as well as components not shown, are possible without departing from the spirit and scope of the present invention. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Not all steps listed in the various figures need be carried out in the specific order described.

What is claimed is:

1. A system having a menstrual blood collection device comprising:
a compressed menstrual tampon includes an elongated generally rod-shaped absorption body having an insertion end and a withdrawal end and a withdrawal string extending from the withdrawal end,
wherein the absorption body has at least one wave-shaped compression line extending continuously from the insertion end to the withdrawal end; and
wherein the compressed menstrual tampon includes a rope that extends from the cotton insert then out the applicator is connected to a pad.

2. The system of claim 1, further comprising: an expandable bag catamenial tampon.

3. The system of claim 1, further comprising: spreading tampon applicators.

4. The system of claim 3, wherein the applicators split into two pieces for ease of removal.

5. The system of claim 2, wherein the catamenial tampon is a soft, conformable, hollow bag.

6. The system of claim 4, wherein the applicators can insert the tampon into the vaginal tract and spreading the tampon open within the vaginal cavity.

7. The system of claim 6, wherein the applicators have an applicator element with a leading end and a trailing end.

8. A menstrual blood collection device comprising:
a compressed menstrual tampon includes an elongated generally rod-shaped absorption body having an insertion end and a withdrawal end and a withdrawal string extending from the withdrawal end,
wherein the absorption body has at least one wave-shaped compression line extending continuously from the insertion end to the withdrawal end; and
wherein the compressed menstrual tampon includes a rope that extends from the cotton insert then out the applicator is connected to a pad.

9. The device of claim 8, further comprising: an expandable bag catamenial tampon.

10. The device of claim 8, further comprising: spreading tampon applicators.

11. The device of claim 10, wherein the applicators split into two pieces for ease of removal.

12. The device of claim 9, wherein the catamenial tampon is a soft, conformable, hollow bag.

13. The device of claim 11, wherein the applicators can insert the tampon into the vaginal tract and spreading the tampon open within the vaginal cavity.

14. The device of claim 13, wherein the applicators have an applicator element with a leading end and a trailing end.
